(19) United States
(12) Patent Application Publication
(10) Pub. No.: US 2026/0262924 A1
(43) Pub. Date: Sep. 10, 2026

(54) CAMERA HEAD FOR ENDOSCOPIC ELECTROSURGICAL PROCEDURES

(71) Applicant: ODYSIGHT.AI LTD., Omer (IL)

(72) Inventors: Amir GOVRIN, Ramat Gan (IL); Yekaterina DLUGACH, Mabuim (IL); Doron KATZIR, Sde Yitzhak (IL)

(21) Appl. No.: 19/166,555

(22) PCT Filed: Mar. 21, 2023
(86) PCT No.: PCT/IL2023/050297

§ 371 Date: Sep. 18, 2025

(51) Int. Cl. H04N 7/10; A61B 1/00; A61B 1/018; A61B 1/05; H04N 23/50; H04N 23/51; H04N 23/52

(52) U.S. Cl. CPC H04N 7/102; A61B 1/00009; A61B 1/00114; A61B 1/018; A61B 1/05; H04N 23/51; H04N 23/52; H04N 23/555; A61B 2562/182

(57) ABSTRACT

Provided herein is a camera head for use in endoscopic electrosurgical procedures, the camera head is configured to, at least partially, reduce interference between electromagnetic radiation associated with electrosurgical procedures and image acquisition. Further provided are endoscopes including the camera head and uses thereof in electrosurgical procedures.

### TECHNICAL FIELD

The present invention relates to a camera head for use in endoscopic electrosurgical procedures, the camera head configured to, at least partially, reduce interference between electromagnetic radiation associated with electrosurgical procedures and image acquisition.

### BACKGROUND

Using a camera for electrosurgical (or electrocautery) procedures is challenging, since the electromagnetic energy generated during the procedure, can interfere with the image acquisition and/or image transmission, causing the obtained images to become distorted and in some cases may also induce camera malfunction.

Built-in protection against interference using metal shielding, such as a metal casing or radiation shield, which absorbs the energy emitted by the electrosurgery procedure, consequently heats, thereby damaging the camera, and may also harm the patient, if used for an extended duration of time. Additionally, metal shielding, cannot cover all portions of the camera, in particular elements which should be transparent, such as lenses, and therefore such shielding may not be completely effective in blocking all of the harmful energy emitted during the electrosurgical procedure.

In addition, intermittent imaging, wherein either the electrosurgical current or the camera is periodically turned off while the other is in use, may not be possible during certain surgical procedures, as it causes interruption in workflow and increase the length of the surgical procedure, and additionally, critical visuals may be missed.

There is thus a need for a camera head for use in endoscopic electrosurgical procedures, to facilitate such procedures while allowing continuous electrosurgical energy along with simultaneous continuous imaging.

### SUMMARY

The present invention in embodiments thereof relates to a camera head for use in endoscopic electrosurgical procedures, wherein the camera head is configured to at least partially reduce interference between electromagnetic radiation associated with the electrosurgical procedures and image acquisition and/or image transfer.

According to some embodiments, the camera head disclosed herein may at least partially shield components of the camera from the electrosurgical associated radiation and/or may enhance continuous image acquisition and/or image transfer with at least partially diminished interference caused by the electrosurgical associated radiation without significant heating of the camera components.

In some embodiments, the camera head disclosed herein may be positioned in a distal end of an endoscope, in particular, an endoscope utilized in electrosurgical procedures. In such embodiments, advantageously, despite the relative proximity between the camera head and the radiation emitting element, both being positioned at the distal end of the endoscope, the advantageous camera head reduces or even eliminates interference between the imaging signals and the electrosurgical radiation, thereby facilitating a continuous and uninterrupted imaged medical procedure.

Some embodiments relate to a camera head for use in endoscopic electrosurgery procedures, the camera head including:

at least one image sensor; and

a serializer module configured to convert one or more image signals acquired by the image sensor to a serial signal configured to be transmitted at a frequency which is different to a frequency of the radiation generated during the electrosurgery procedure.

Some embodiments relate to a camera head for use in endoscopic electrosurgery procedures, the camera head including:

at least one image sensor; and

an electric circuit board associated with the image sensor, wherein the electric circuit board is coated with a conducting layer for shielding the electric circuit board from signals generated by the electrosurgical process,

wherein at least a distal portion of the electric circuit board is configured to deviate at an angle α relative to a longitudinal axis defined by the center of the corresponding lens,

wherein the angle of the image sensor is configured to at least partially prevent, interference of image acquisition and/or transfer by radiation generated during the electrosurgery procedure.

Some embodiments relate to a camera head for use in endoscopic electrosurgery procedures, the camera head including:

at least one image sensor and at least one corresponding lens;

a casing at least partially composed of a non-conductive material; and

a coating on a surface of the casing, wherein the casing encloses at least the image sensor and is configured to at least partially prevent interference of image acquisition and/or transfer by radiation generated during the electrosurgery procedure.

According to some embodiments, the radiation generated during the electrosurgery procedure may be electromagnetic radiation. According to some embodiments, the electromagnetic radiation generated during the electrosurgery procedure may have a radiofrequency radiation (RF) in the range of about 0.1 to about 4 MHz.

According to some embodiments, the serial signal generated by the serializer module may be transmitted at a frequency in the range of about 2 to about 8 GHZ.

According to some embodiments, the camera head may be associated with a distal portion of a transmission cable configured to transfer signals from the image sensor to a proximal control unit.

According to some embodiments, the camera head may further include a decoder module at the proximal end of the transmission cable, or at the control unit, wherein the decoder module is configured to convert the transmitted serialized signal to a non-serialized signal.

According to some embodiments, the serialization of the signals by the serializer module and/or the deserialization of the signals by the module may make use of a Mobile Industry Processor Interface (MIPI), a Low Voltage Differential Signaling (LVDS), Display port, HDMI (High-Definition Multimedia Interface), SDI (Serial Digital Interface), HD-SDI (High Definition Serial Digital Interface), DVI (Digital Visual Interface), FPD-Link and/or a GMSL (Gigabit Multimedia Serial Link) protocol.

According to some embodiments, the camera head may further include a housing at least partially composed of a non-conductive material, wherein the non-conducting material may be a thermoset and or thermoplastic and/or a ceramic materials.

According to some embodiments, the housing may further include a coating on a surface of the casing, wherein the coating is provided on an inner surface of the casing. According to some embodiments, the coating may be a layer of a conductive polymer, a metal, or a combination thereof, e.g., copper, gold, silver, iron, aluminum, nickel, or any combinations thereof. According to some embodiments, the metal coating may be coated onto the casing by an electroplating process, wherein the coating has a thickness in the range of between about 0.1 μm to about 30 μm. According to some embodiments, the metal coating may have a low thermal capacity due to a thickness thereof.

According to some embodiments, the camera image sensor may be positioned on an electronic circuit board associated with the image sensor, wherein at least a distal portion of the circuit board may be configured to deviate at an angle α relative to a longitudinal axis of a corresponding lens. According to some embodiments, the angle α may be in the range between about 15° to about 180°.

According to some embodiments, the camera head may further include at least one of an illumination source, a communication unit and a power unit or any combination thereof.

According to some embodiments, the electrosurgical procedure may be selected from: cutting, coagulating, dissecting, fulgurating, ablating and/or shrinking tissue.

According to some embodiments, an endoscope may include the camera head and at least one working channel for electrosurgery tools.

Some embodiments relate to a method for at least partially preventing interference of image acquisition and/or image transferring in a camera head in an endoscopic electrosurgery procedure, the method comprising:

providing at least one image sensor configured to acquire one or more images;

converting one or more image signals acquired by the image sensor during the endoscopic electrosurgical procedure to a serial signal using a serializer module configured for transmitting at a frequency which is different to a frequency of the radiation generated during the electrosurgery procedure,

thereby at least partially preventing interference of image acquisition and/or transfer by radiation generated during the electrosurgery procedure.

Some embodiments relate to a method for at least partially preventing interference of image acquisition and/or image transferring in a camera head in an endoscopic electrosurgery procedure, the method including:

encasing at least one image sensor and at least one corresponding lens; and

positioning a coated circuit board associated with the image sensor, at an angle α relative to a longitudinal axis defined by the center of the corresponding lens, wherein the circuit board is coated with at least one conductive layer,

thereby at least partially preventing interference of image acquisition and/or image data transfer by radiation generated during the electrosurgery procedure.

According to some embodiments, the method may include converting the transmitted serialized signal to a non-serialized signal using a decoder module at the proximal end of a transmission cable or at a control unit.

Some embodiments relate to a method at least partially preventing interference of image acquisition and/or image transferring in a camera head in an endoscopic electrosurgery procedure, the method comprising:

encasing at least one image sensor and at least one corresponding lens in a casing at least partially composed of a non-conductive material coated by a conductive material; and

acquiring and transmitting images by the image sensor during an electrosurgical procedure,

at least partially preventing interference of image acquisition and/or transfer by radiation generated during the electrosurgery procedure by the coated casing.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1: Schematic diagram of a camera head with a conversion module (“serializer”) in accordance with some embodiments of a first aspect of the invention;

FIG. 2: Schematic diagram of a camera head with a coated casing, in accordance with some embodiments of a second aspect of the invention;

FIG. 3: Schematic diagram of a camera head with an angled circuit board, in accordance with some embodiments of a third aspect of the invention;

FIG. 4: Schematic diagram of a camera head, in accordance with some embodiments combining the first, second and third aspect of the invention;

FIG. 5: Schematic view of a front view of a distal tip of an endoscope comprising a camera head, in accordance with some embodiments;

FIG. 6: Schematic view of an endoscope comprising a camera head and a channel for an electrosurgical device, in accordance with some embodiments;

FIG. 7 shows a schematic diagram of an electrosurgical endoscopic system having a distal camera head and a proximal control unit, in accordance with some embodiments.

FIG. 8: Flow chart of a method for at least partially reducing interference between electromagnetic radiation associated with the electrosurgical procedures and image acquisition and/or image transfer in endoscopic electrosurgery procedures, in accordance with some embodiments of a second aspect of the invention;

FIG. 9: Flow chart of a method for at least partially reducing interference between electromagnetic radiation associated with the electrosurgical procedures and image acquisition and/or image transfer in endoscopic electrosurgery procedures, in accordance with some embodiments of a third aspect of the invention; and

FIG. 10: Flow chart of a method for at least partially reducing interference between electromagnetic radiation associated with the electrosurgical procedures and image acquisition and/or image transfer in endoscopic electrosurgery procedures, in accordance with some embodiments of a first aspect of the invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

In the following description, numerous details are set forth for the purpose of explanation. However, one of ordinary skill in the art will realize that the invention may be practiced without the use of these specific details.

According to some embodiments, there is provided herein a camera head for use in endoscopic electrosurgical procedures. In some embodiments, the camera head may be configured to associate and/or may be part of an imaging device, such as, an endoscope or visualization probe. According to some embodiments, the camera head may be included within an endoscope or visualization probe. In some embodiments, the camera head may operate in an electrosurgical environment, for example being provided in an electrosurgical endoscope which may include one or more channels for electrosurgery tools, e.g., working channel. According to some embodiments, the camera head disclosed herein may be configured to, at least partially, reduce interference between electromagnetic radiation associated with electrosurgical procedures and image acquisition, image processing and/or image transmission performed simultaneously with an electrosurgical procedure. According to some embodiments, interference may be reduced without significant heating of the camera components, e.g., without harming the camera components and/or without causing harm to the patient by heat.

According to some embodiments, the camera head disclosed herein may facilitate electrosurgical procedures with continuous electrosurgical current together with simultaneous continuous imaging. According to some embodiments, the camera head may be configured to reduce, inhibit and/or prevent distortion of the images acquired and/or transmitted by the image sensor by the surrounding electrosurgical radiation. According to some embodiments, the camera head may be configured to prevent heating of the image sensor while shielding the image sensor from electromagnetic interference. According to some embodiments, the camera head may be configured to reduce, inhibit and/or prevent distortion of the images acquired and/or transmitted by the image sensor by the surrounding electrosurgical radiation without the use of a large amount of metal shielding, which may result in overheating of the device and/or injury to the patient due to absorption of heat generated during the electrosurgical process.

As disclosed herein, according to some embodiments, the camera head may facilitate reducing and/or even eliminating electromagnetic radiation interference generated by an electrosurgical procedure via mechanical, physical and/or electronical means.

A first aspect of some embodiments of the invention relate to a camera head including an image sensor and an internal conversion module. According to some embodiments, the conversion module may include a serializer module, e.g., which is configured to convert a parallel data stream into a serial data stream, and/or a decoder module, which may perform the opposite function, e.g., configured to convert a serial data stream into parallel data.

For example, a serializer module may convert a block of digital data into a bit stream which may be transmitted over a serial communication channel such as a wire or optical fiber.

An optional advantage of using a serializer is that it may reduce the number of wires required for data transmission and/or may increase the speed and distance of communication. On the receiving end, a corresponding decoder module may convert the bit stream back into the original parallel data. This allows the original digital data to be reconstructed at the receiving end without any loss of information and/or corruption of the data.

According to some embodiments, the conversion module may be configured for converting digital data into a bit stream which may be transmitted over a serial communication channel. In some embodiments, the data may be transmitted over a serial communication channel at a frequency which is unaffected by electromagnetic radiation generated during an electrosurgical procedure. In some embodiments, the image signals may be converted within the camera head before transmission of the signals. In some embodiments, a serializer module may be provided within the camera head. According to some embodiments, the serializer module may be located at a close distance to the image sensor. Optionally, the image sensor and the serializer module may be provided on a same circuit board. In some embodiments, the distance between the image sensor and the serializer module may be less than about 40 mm, i.e., between about 0.1 mm to about 40 mm, between about 0.1 mm to about 30 mm, between about 0.1 mm to about 20 mm, between about 1 mm to about 10 mm, between about 1 mm to about 5 mm and/or any intermediate range.

According to some embodiments, the differences in frequencies may prevent signal interferences. According to some embodiments, when two or more signal have different frequencies, they may occupy different portions of the electromagnetic spectrum. According to some embodiments, two or more different signals may be less likely to interfere with each other if they are not competing for the same bandwidth.

According to some embodiments, electromagnetic interference may occur when two signals share the same frequency range and overlap with each other, which may result in distortion and/or complete loss of the signal. According to some embodiments, when signals have different frequencies, they may coexist and may be transmitted without interference, as each signal may be contained within its own frequency band.

According to some embodiments, the transmitted frequency may be different to the frequency of the radiofrequency radiation generated during the electrosurgery procedure e.g., a higher frequency. According to some embodiments, the serial signal generated by the serializer module may be transmitted at a frequency in the range of between about 1 to about 10 GHz, between about 2 to about 8 GHz, between 3 to about 6 GHz, between about 1 to about 3 GHz and/or any intermediate range.

According to some embodiments, the electromagnetic radiation generated during the electrosurgery procedure may be a radiofrequency radiation in the range between about 0.1 to about 4 MHz, between about 0.1 to about 3 MHz, between about 0.5 to about 2 MHz, between about 0.5 to about 1 MHz. According to some embodiments, the electrosurgical procedure may be selected from: cutting, coagulating, dissecting, fulgurating, ablating, vaporizing, destroying and/or shrinking tissue. According to some embodiments, the frequency used may depend on the specific device being used and/or the intended application. For example, in electrocautery a higher frequency is typically used around about 3 to about 4 MHz to achieve a more precise and controlled cauterization of tissue, while in electrosurgery, a lower frequency is often used around 100-500 kHz to cut and coagulate tissue simultaneously.

According to some embodiments, the camera head may be associated with a distal portion of a transmission cable. According to some embodiments, the transmission cable may be configured to transfer signals from the image sensor to a proximal control unit. According to some embodiments, the proximal control unit may be located on and/or in the vicinity of the handle of the endoscope. In other embodiments, the transmission cable may be connected at the proximal end to a console for viewing the images taken by the camera head.

According to some embodiments, the transmission of signals at a high frequency may not only allow minimal interference and/or disturbances by the electromagnetic radiation, but may also provide a potential benefit of facilitating transferring the imaging signals over longer distances (i.e., using longer cables), thereby allowing easier and safer operation of the imaging device associated with the camera head (e.g., an endoscope) by a user.

According to some embodiments, the transmission cable may have a length in the range between about 0.5 m to about 5 m, between about 0.8 m to about 1 m, between about 1 m to about 2.5 m, and/or between about 2.5 m to about 5 m.

According to some embodiments, a decoder module, such as a deserializer module, may be located at the proximal end of the transmission cable to decode the image signals. According to some embodiments, a decoder module may be located at a connector to the control unit. Optionally, the connector module includes a circuit board on which the decoder may also be provided. According to some embodiments, the decoder module may be configured to convert the transmitted serialized signal to a non-serialized signal.

According to some embodiments, the serialization of the signals by the conversion module (serializer module) and/or the deserialization of the signals by the decoder module may make use of a Mobile Industry Processor Interface (MIPI), Low Voltage Differential Signaling (LVDS) protocol, Display port, HDMI (High-Definition Multimedia Interface), SDI (Serial Digital Interface), HD-SDI (High Definition Serial Digital Interface), DVI (Digital Visual Interface), FPD-Link, GMSL (Gigabit Multimedia Serial Link) protocol and/or any other suitable signaling protocol.

A second aspect of some embodiments of the invention relates to a camera head provided within an internal shield configured to at least partially protect at least the image sensor of the camera head by physically, mechanically and/or electronically shielding the camera head from radiation (such as, radiofrequency (RF) electromagnetic radiation) generated during the electrosurgical procedure. According to some embodiments, heat generated during the electrosurgical procedure may not significantly heat the internal shield and/or may not harm the camera head components and/or the patient by heating.

According to some embodiments, the camera head, and/or portions thereof, may be provided in a casing (housing) composed at least in part of ceramic, plastic or a polymer. Non-limiting examples of suitable materials for a casing are polycarbonate, polypropylene, polyethylene, ceramic and/or a combination thereof.

According to some embodiments, the casing (housing) may be coated with an electrically conductive material, such as an electrically conductive metal and/or an electrically conductive polymer. According to some embodiments, the coating may be configured to, at least partially, shield the image sensor from radiation generated during an electrosurgical procedure. According to some embodiments, the coating may be one or more layers of one or more metals. Non-limiting examples of suitable coating materials are metals which may be selected from the group consisting of copper, gold, silver, iron, aluminum, nickel, and/or any combination thereof.

According to some embodiments, the thickness of the coating may be sufficient to shield the camera head from electromagnetic interference while the thermal capacity of the shield may be small so as not to cause injury to the camera components and/or patient. According to some embodiments, the coating may have a thickness in the range between about 0.1 μm to about 1 μm, between about 1 μm to about 5 μm, between about 5 μm to about 25 μm, between 10 μm to about 30 μm, and/or between about 25 μm to about 50 μm.

In some embodiments, the casing may be coated on an inner surface thereof. According to some embodiments, an inner coating may prevent harming the patient with the heat absorbed by the electrically conductive material. In other embodiments, the casing may be coated on an outer surface thereof. According to some embodiments, the coating may be thin enough so as not to cause harm and/or injury to the patient. According to some embodiments, one or more additional layers may separate between the casing and the patient.

According to some embodiments, the coating may be applied onto the casing by an electroplating process. According to some embodiments, the metal coating may be applied to a molded and/or cast and/or machined casing. According to some embodiments, the metal coating may be applied prior to molding and/or casting the casing.

According to some embodiments, the camera head for use in endoscopic electrosurgery procedures may include one or more image sensors, and optionally, at least one corresponding lens, a casing composed at least partially of a non-conductive material and a coating on a surface of the casing. According to some embodiments, the casing may enclose at least the image sensor. In some embodiments, the casing may enclose a conversion module. According to some embodiments, the casing and/or coating may be configured to, at least partially, shield the image sensor from radiation generated during an electrosurgery procedure.

According to some embodiments, the one or more image sensors may be an optical camera, charge-coupled device (CCD), an active-pixel sensor (CMOS sensor), digital single-lens reflex camera (DSLR), Indium gallium arsenide (InGaAs) camera, Backside Illuminated (BSI) camera, Complementary Metal Oxide Semiconductor (CMOS) camera, thermal imaging camera, ultrasound imaging sensor, etc. Each possibility is a separate embodiment.

According to some embodiments, the image sensor may be protected by an associated lens. According to some embodiments, the lens may be a single and/or dual lens. Non-limiting examples of materials lens may be composed of are glass, transparent silicon, crystal lens, quartz lens, a transparent polymeric material such as acrylic, polycarbonate, Columbia Resin #39 (CR-39), cyclo-olefin polymer (COP), etc. Each possibility is a separate embodiment. According to some embodiments, one or more lenses may be coated. Non-limiting examples of lens coatings are an anti-reflective coating, IR-blocking coating, anti-fog coating, hydrophobic coating, oleophobic coating, etc. According to some embodiments, coating processes and/or materials may add electromagnetic interference (EMI) shielding to the one or more lenses. Additionally, according to some embodiments, coatings may be suitable for predictive maintenance cameras.

According to some embodiments, coating processes for coating a lens and/or a casing may be selected from common coating processes, such as, an electrochemical process, physical vapor deposition (PVD) (e.g., of aluminum, copper, nickel, gold, etc.), sputtering (e.g., of aluminum, copper, silver, etc.), electroplating (e.g., of copper, nickel, gold, etc.), conductive polymer coatings (e.g., polyaniline, polypyrrole, poly(3,4-ethylenedioxythiophene) (PEDOT), etc.), adhesive thin metal meshes (e.g., made from materials such as, copper, silver, aluminum, etc.).

According to some embodiments, the casing may be at least partially composed of a non-conducting material. According to some embodiments, the non-conducting material may be a thermoplastic, thermoset plastic, a ceramic, and/or a combination thereof. According to some embodiments, the non-conducting material may be non-toxic and/or anti-allergenic, and/or biocompatible.

According to some embodiments, the camera head may further include one or more illumination sources. According to some embodiments, an illumination source may be a light emitting diode (LED) and/or one or more optical fibers e.g., e.g., the lighting source may inside the casing and may be transmitted by light guides. According to some embodiments, the illumination source may be mounted on the exterior and/or the interior of the casing. Preferably, the illumination source may be mounted on the interior of the casing.

According to some embodiments, the distal surface of the casing, facing the imaged portion, may be transparent to facilitate light to pass therethrough. According to some embodiments, the distal surface of the casing may not be coated with an electrically conductive material, which may be non-transparent. Alternatively, and/or additionally, the distal surface of the casing may be constructed of a translucent material to provide adequate shielding.

In other embodiments, the illumination source may be provided external to the casing and may be transmitted using one or more optical fibers, e.g., the lighting source may be in the proximal part of the device and the light may be transmitted by optical fibers to the camera head. According to some embodiments, the external illumination source may be mounted on a distal surface of the casing. According to some embodiments, the external illumination source may be mounted on and/or in the housing of an endoscopic head.

According to some embodiments, the camera head may include a communication unit, a power unit, one or more image sensors, one or more illumination sources, a serializer, a lens, a casing, and/or any combination thereof.

According to some embodiments, the camera head may include at least one image sensor, a casing at least partially composed of a non-conductive material, and an electric circuit board associated with the image sensor.

According third aspect of some embodiments, a camera head may be provided with one or more image sensors mounted on an electric circuit board, such as a printed circuit board (PCB). In some embodiments, the circuit board may be composed of one or more layers. According to some embodiments, at least one of the layer of the electric circuit board may include a thin metal layer for shielding the electric circuit board from signals generated by the electrosurgical process. According to some embodiments, the layer coating the electric circuit board may have a thickness in the range between about 0.1 μm to about 1 μm, between about 1 μm to about 5 μm, between about 5 μm to about 25 μm, between 10 μm to about 30 μm, and/or between about 25 μm to about 50 μm.

According to some embodiments, the at least a distal portion of the electric circuit board may be configured to deviate at an angle α relative to a longitudinal axis defined by the center of a corresponding lens. According to some embodiments, the angle of the electric circuit board may be configured to at least partially shield the electric circuit board from radiation generated during the electrosurgery procedure and entering through the lens of the camera head.

Additionally, and/or alternatively, in some embodiments, the camera head may be constructed such that the image sensor may be deviated (i.e., positioned at an angle) relative to the position of a corresponding lens at the distal end of the camera head, thereby at least partially reducing direct interference, radiation entering from the lens position and/or opening from reaching the image sensor. According to some embodiments, the image sensor may be mounted on an electric circuit board which may be configured at an angle α relative to a longitudinal axis defined by the center of the corresponding lens.

According to some embodiments, the longitudinal axis may be parallel to the center of the lens. According to some embodiments, the longitudinal axis may be the longitudinal axis of the endoscope in which the camera head is provided. According to some embodiments, the longitudinal axis may be perpendicular to the center of the lens. According to some embodiments, angle α may be in the range between about 0° to about 180°, between about 5° to about 120°, between about 10 to about 100°, between about 15° to about 90°, between about 20° to about 75°, and/or between about 15° to about 180°.

According to some embodiments of the invention, various embodiments of the invention, e.g., the first, second and/or third aspects may be combined. For example, the encoding module described with respect to embodiments of the first aspect of the invention may be provided within a casing as described with respect to embodiments of the second aspect of the invention. In addition, at least a distal portion of the electric circuit board on which the image sensor and/or encoding module may be mounted as described with respect to embodiments of the first aspect of the invention may be configured to deviate as described with respect to embodiments of the third aspect of the invention and/or may be enclosed within a casing as described with respect to embodiments of the second aspect.

Some embodiments relate to a camera head for use in endoscopic electrosurgery procedures including:
a) at least one image sensor;
b) at least one lens corresponding to the image sensor, and
c) one or more of:
   i) a casing at least partially composed of a non-conductive material, and a coating on an inner surface of the casing wherein the casing encloses at least the image sensor, and is configured to at least partially shield the image sensor from radiation generated during the electrosurgery procedure;
   ii) a distal portion of an electric circuit board associated with the image sensor configured to deviate at an angle α relative to a longitudinal axis defined by the center of the corresponding lens; and/or
   iii) a serializer module configured to convert one or more image signals acquired by the image sensor to a serial signal configured to be transmitted at a frequency which is different than a frequency of the radiation generated during the electrosurgery procedure.

Some embodiments relate to a method for at least partially shielding an image sensor in a camera head for use in endoscopic electrosurgery procedures including:
a) providing at least one image sensor and corresponding lens;
b) one or more of:
   i) encasing the at least one image sensor in a casing at least partially composed of a non-conductive material, and coating an inner surface of the casing with a conductive material, thereby at least partially shielding the image sensor from radiation generated during the electrosurgery procedure,
   ii) positioning a circuit board (e.g., PCB) associated with the image sensor at an angle α relative to a longitudinal axis defined by the center of a corresponding lens and mounting the image sensor on the angled circuit board, and
   iii) converting one or more image signals acquired by the image sensor to a serial signal using a serializer module configured for transmitting at a frequency which is different than a frequency of the radiation generated during the electrosurgery procedure, and transmitting the serialized signal over a transmission cable,
      thereby at least partially shield the image sensor from radiation generated during the electrosurgery procedure.

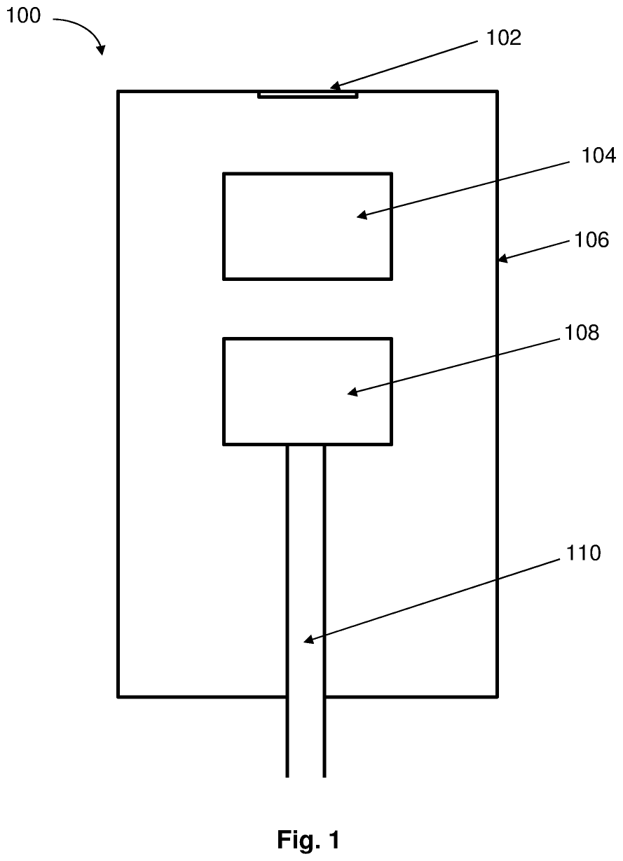

Reference is now made to FIG. 1 which shows a schematic diagram of a camera head with a serializer module, in accordance with some embodiments of the first aspect of the invention. According to some embodiments, a camera head 100 for use in endoscopic electrosurgery procedures may include within a housing 106 at least one image sensor 104, at least one corresponding lens 102, and a conversion module 108. Non-limiting examples of image sensors are optical camera, charge-coupled device (CCD), an active-pixel sensor (CMOS sensor), digital single-lens reflex camera (DSLR), Indium gallium arsenide (InGaAs) camera, Backside Illuminated (BSI) camera, Complementary Metal Oxide Semiconductor (CMOS) camera, thermal imaging camera, ultrasound imaging sensor, and the like.

According to some embodiments, the conversion module may comprise a serializer module 108 which may be configured to convert one or more image signals acquired by the image sensor 104 to a serial signal configured to be transmitted at a frequency. According to some embodiments, the transmitted frequency may be different to the frequency of the radiation generated during an electrosurgery procedure, e.g., a higher frequency.

For example, an image sensor may provide a signal using a Mobile Industry Processor Interface (MIPI), a Low Voltage Differential Signaling (LVDS) protocol, Display port, HDMI (High-Definition Multimedia Interface), SDI (Serial Digital Interface), HD-SDI (High-Definition Serial Digital Interface), DVI (Digital Visual Interface), FPD-Link, or GMSL (Gigabit Multimedia Serial Link) protocol. A serializer module may convert the signal (e.g., 16 signals) into one bidirectional serial line. This may increase the frequency at which the signals are transmitted (e.g., to about 2-6 GHz, for example 3 GHz) and the image sensor signals may not interfere with the electrosurgical frequency which is lower (e.g., up to about 400 MHz). Optionally, as the signals are at a high frequency, even if there is some disruption from the electrosurgical signals this does not affect the image signals.

Additionally, in some embodiments, the camera head may be associated with a serializer module, which allows processing and/or converting the imaging signals obtained by the image sensor into a bidirectional serial line, to result in a different frequency (e.g., higher frequency) at which the serialized signals are transmitted from the camera head (for example, via a corresponding cable) towards a proximal control unit. Such an increase in frequency of signal transmittance may result in reduction and/or elimination of the interference of the radiation frequency associated with the electrosurgical procedure (e.g., at a lower frequency). Thus, in such a setting, the serialized, high frequency signal transferred and/or transmitted from the camera head may not be affected by the electrosurgical procedure, and the serialized signal can be re-converted to distinct imaging signals by a suitable destabilizer module in the proximal end of the cable and/or in a control unit connected thereto.

According to some embodiments, the serial signal generated by the serializer module may be transmitted at a frequency in the range of between about 1 to about 10 GHz, between about 2 to about 8 GHz, between 3 to about 6 GHz, between about 1 to about 3 GHZ and/or any intermediate range.

According to some embodiments, the electromagnetic radiation generated during the electrosurgery procedure may be a radiofrequency radiation in the range between about 0.1 to about 4 MHz, between about 0.1 to about 3 MHz, between about 0.5 to about 2 MHz, between about 0.5 to about 1 MHz.

According to some embodiments, the camera head may be associated with a distal portion 110 of a transmission cable. According to some embodiments, the transmission cable may be configured to transfer signals from the image sensor to a proximal control unit. According to some embodiments, the proximal control unit may be located on and/or in the vicinity of the handle of the endoscope. According to some embodiments, the transmission cable may be connected at the proximal end to a console having a display on which the images may be displayed. According to some embodiments, a decoder module may be located at the proximal end of the transmission cable. Optionally, a decoder module may convert the signals back to a Mobile Industry Processor Interface (MIPI), a Low Voltage Differential Signaling (LVDS) protocol, Display port, HDMI (High-Definition Multimedia Interface), SDI (Serial Digital Interface), HD-SDI (High Definition Serial Digital Interface), DVI (Digital Visual Interface), FPD-Link, or GMSL (Gigabit Multimedia Serial Link) protocol.

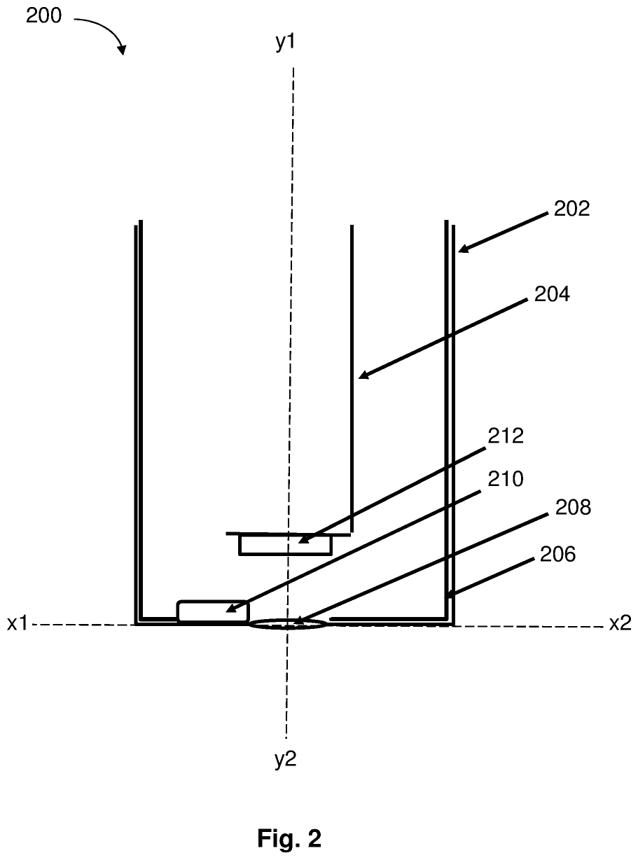

Reference is now made to FIG. 2 which shows a schematic diagram of a camera head with a coated casing, in accordance with some embodiments of a second aspect of the invention. According to some embodiments, the camera head 200 may include a casing 202 enclosing an imaging sensor 212. According to some embodiments, the casing may be composed of a non-conducting material. According to some embodiments, the non-conducting material may be a thermoplastic and/or thermoset plastic, a ceramic, and/or a combination thereof.

According to some embodiments, the casing 202 may be coated 206 on one or more surfaces. According to some embodiments, the casing may be coated with an electrically conductive material, such as an electrically conductive metal and/or an electrically conductive polymer. According to some embodiments, the coating may be configured to, at least partially, shield the image sensor from radiation generated during an electrosurgical procedure. According to some embodiments, the coating may include one or more layers. According to some embodiments, one or more additional layers may separate between the casing and the patient. In some embodiments, the casing may be coated on an inner surface and/or an outer surface thereof. According to some embodiments, the coating may be thin enough so as not to cause harm and/or injury to the patient. According to some embodiments, the coating may have a thickness in the range between about 0.1 μm to about 1 μm, between about 1 μm to about 5 μm, between about 5 μm to about 25 μm, between 10 μm to about 30 μm, and/or between about 25 μm to about 50 μm.

According to some embodiments, all sides of the casing 202 may be coated with a coating 206 except for the lens 208 and/or an illumination source 210. According to some embodiments, an illumination source may be a light emitting diode (LED) and/or one or more optical fibers. According to some embodiments, lens 208 may be transparent. Non-limiting examples of materials lens may be composed of are glass, transparent silicon, crystal lens, quartz lens, a transparent polymeric material such as acrylic, polycarbonate, Columbia Resin #39 (CR-39), cyclo-olefin polymer (COP), etc.

According to some embodiments, the cable and/or sections thereof may be coated. According to some embodiments, the coating may be a protective coating, a hydrophobic coating, a radiofrequency blocking coating, etc.

According to some embodiments, the coating may be a layer of a metal, e.g., copper, gold, silver, iron, aluminum, nickel, and/or any combination thereof. According to some embodiments, the metal coating may be coated onto the casing by an electroplating process. According to some embodiments, the coating may be configured to at least partially shield the image sensor from radiation generated during an electrosurgery procedure. According to some embodiments, the coating may be configured to at least partially prevent interference of image acquisition and/or transfer by radiation generated during an electrosurgery procedure.

According to some embodiments, the metal comprising the coating may be considered toxic and may not be permitted to be in contact with the human body. According to some embodiments, the casing may prevent contact between the inner metal coating and the human body. According to some embodiments, the non-conducting casing may prevent and/or reduce heat absorption and/or heat transfer from an electrosurgery procedure, thereby preventing and/or reducing harm to the human body.

According to some embodiments, the small thermal mass of the coating may prevent heating as a result of the radiation. According to some embodiments, small masses of metal may not absorb much RF (Radio Frequency) radiation and are less affected by it in terms of heat because of their small size and low electrical conductivity. For example, when RF radiation encounters a metallic object, it may induce an electrical current within the object. The amount of heat generated in the metal due to this induced current may depend on the electrical conductivity of the metal and the amount of RF energy absorbed. In general, highly conductive metals, such as copper and aluminum, may absorb a significant amount of RF energy and may heat up quickly. However, small masses of metal may have a low surface area, and hence, their ability to absorb RF radiation may be limited. Additionally, they may not have enough bulk to retain much heat, so the heat generated by the absorbed RF energy may be quickly dissipated into the surroundings.

According to some embodiments, the illumination source 210 may be located on an internal and/or external surface of the casing. According to some embodiments, the distal surface of casing 202, facing the imaged portion, may be transparent to facilitate light to pass therethrough. According to some embodiments, the distal surface of the casing may not be coated with an electrically conductive material, which may be non-transparent. Alternatively, and/or additionally, the distal surface of the casing may be constructed of a translucent material.

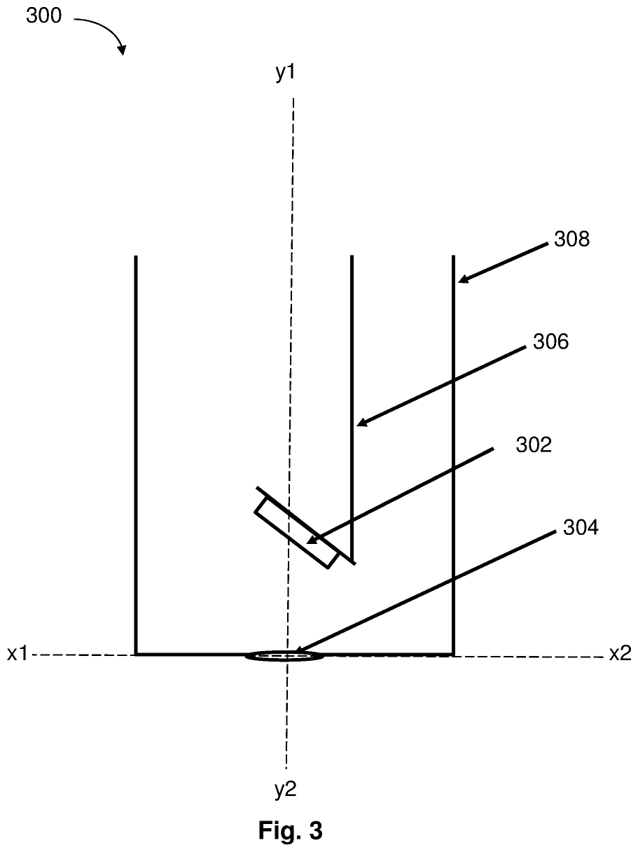

Reference is now made to FIG. 3 which shows a schematic diagram of a camera head with an angled (bent, deviated) distal portion of an electric circuit board, in accordance with some embodiments. According to some embodiments, the camera head 300 for use in endoscopic electrosurgery procedures, the camera head may include a housing 308 enclosing at least one image sensor 302 and at least one corresponding lens 304, and a circuit board 306 (such as a PCB) associated with the image sensor. In some embodiments, the circuit board may be composed of one or more layers. According to some embodiments, at least one of the layer of the electric circuit board may include coating, e.g., a thin metal layer, for shielding the electric circuit board from signals generated by the electrosurgical process. According to some embodiments, at least a distal portion of the electric circuit board may be configured to be deviated at an angle to at least partially shield the image sensor due to the coating on the electric circuit board. According to some embodiments, the layer coating the electric circuit board may have a thickness in the range between about 0.1 μm to about 1 μm, between about 1 μm to about 5 μm, between about 5 μm to about 25 μm, between 10 μm to about 30 μm, and/or between about 25 μm to about 50 μm.

According to some embodiments, at least a distal portion of the electric circuit board 306 may be configured to deviate at an angle α relative to a longitudinal axis defined by the center of the corresponding lens 304. According to some embodiments, at least a distal portion of the electric e circuit board 306 may be bent, deviated and/or deflected at an angle α relative to a longitudinal axis defined by the center of the corresponding lens 304. According to some embodiments, the longitudinal axis may be along axes x1-x2 and/or y1-y2, as shown in FIG. 3.

According to some embodiments, the angle of the circuit board may be configured to, at least partially, prevent interference of image acquisition and/or transfer by radiation generated during an electrosurgery procedure. According to some embodiments, the amount of incoming disruptive radiofrequency radiation from an endoscopic electrosurgery procedure to which an image sensor may be exposed may be reduced.

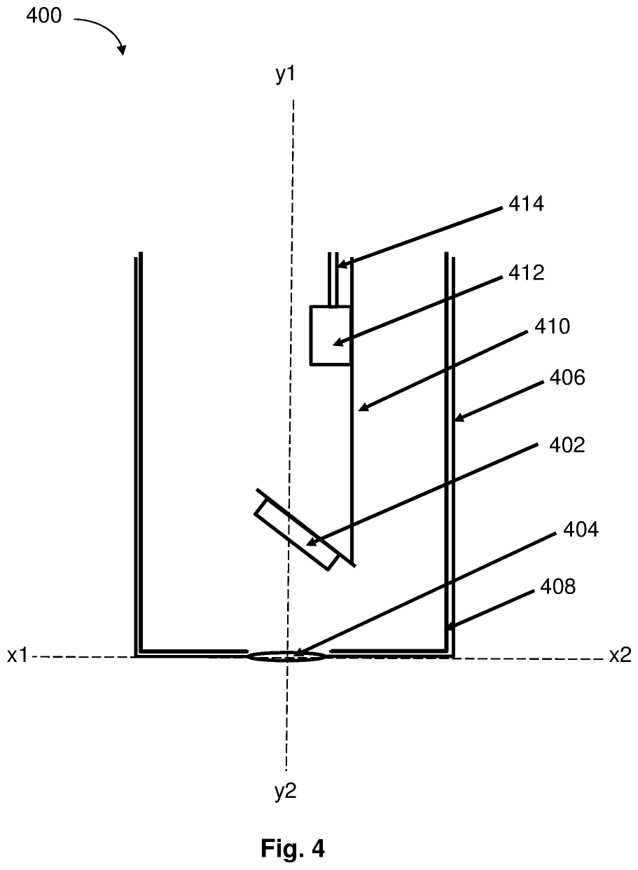

Reference is now made to FIG. 4, showing a schematic diagram of a camera head, in accordance with some embodiments combining different aspects of the invention. Some embodiments relate to a camera head 400 for use in endoscopic electrosurgery procedures including at least one image sensor 402, at least one lens 404 corresponding to the image sensor 402, a casing 406 at least partially composed of a non-conductive material, wherein the casing encloses at least the image sensor 402, and a combination of one or more of: i) an electric conductive coating 408 on an inner surface of the casing 406 configured to at least partially prevent interference of image acquisition and/or transfer by radiation generated during an electrosurgery procedure; ii) at least a distal portion of the electric circuit board 410 (e.g., PCB) associated with the image sensor 402 and configured be deviated at an angle α (i.e. bent or deviated) relative to a longitudinal axis defined by the center of the corresponding lens 404; and iii) a conversion module, e.g. serializer module 412, configured to convert one or more image signals acquired by the image sensor 402 to a serial signal configured to be transmitted at a frequency which is different than the frequency of the radiation generated during the electrosurgery procedure (e.g., higher frequency), and converting the transmitted serialized signal to a non-serialized signal using a decoder module at the proximal end of a transmission cable 414 or at a control unit; thereby at least partially prevent interference of image acquisition and/or transfer by radiation generated during an electrosurgery procedure.

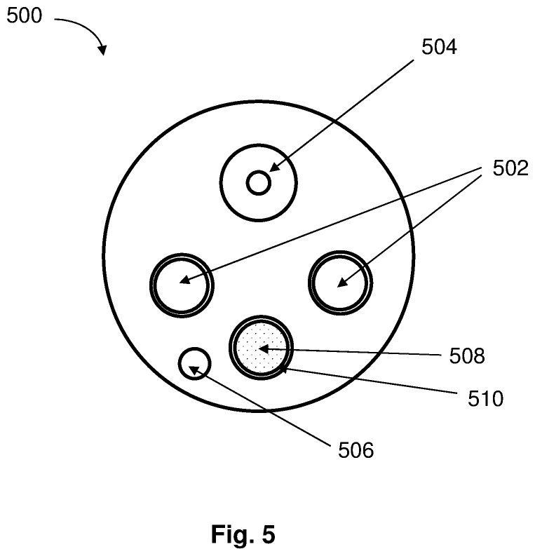

Reference is now made to FIG. 5, which shows a schematic diagram of a front view of an endoscopic head, in accordance with some embodiments. Some embodiments relate to an endoscopic head 500 for use in endoscopic electrosurgery procedures including a camera head (not shown) as described herein. According to some embodiments, the endoscopic head may include one or more illumination sources 502, a working channel 504 for one or more electrosurgery tools, an air and/or water nozzle and/or suction 506, and an image sensor 508 covered by a lens 510. Optionally, the camera head may be enclosed in a non-conducting casing coated on an inner surface with a conducting material. Optionally, the image sensor may be located on a circuit board positioned at an angle to an axis of the lens. Optionally, the camera head may include a serializer module configured to change the frequency of a signal from the image sensor.

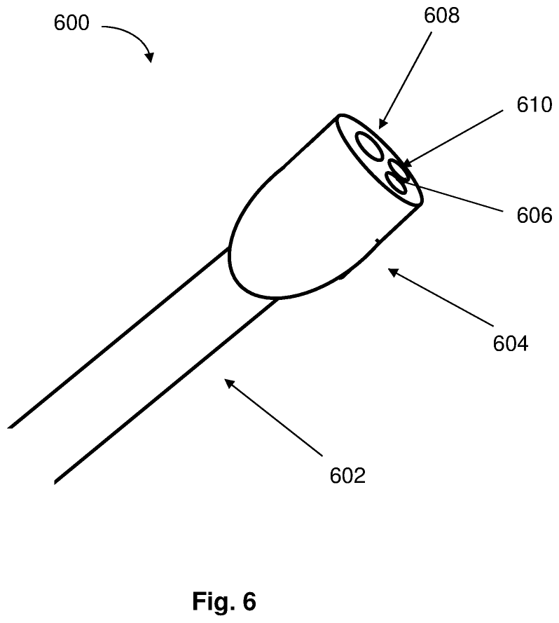

Reference is now made to FIG. 6, which shows a schematic diagram of an electrosurgical endoscopic system having a camera head, in accordance with some embodiments. According to some embodiments, an endoscopic system 600 may include a shaft 602 and a distal end, holding an endoscopic head 604 including a camera head 606, as disclosed herein, one or more illumination sources 610, and one or more working channels 608, to facilitate electrosurgical procedures. According to some embodiments, the shaft 602 may include one or more working channels for endosurgical tools (such as, electrosurgical tools), one or more channels for the transmission of air and/or water, one or more transmission cables for transmission of signals to and/or from one or more units within the endoscopic distal end, e.g., an image sensor, illumination source, electrosurgical tool, serializer module, etc. In some embodiments, the camera is included within an endoscope and the one or more endosurgical tools are configured to be inserted to the region of interest within the subject body, via separate, distinct ports.

According to some embodiments, an image sensor may provide a signal using a Mobile Industry Processor Interface (MIPI) a Low Voltage Differential Signaling (LVDS) protocol, Display port, HDMI (High-Definition Multimedia Interface), SDI (Serial Digital Interface), HD-SDI (High Definition Serial Digital Interface), DVI (Digital Visual Interface), FPD-Link, or GMSL (Gigabit Multimedia Serial Link)protocol. A serializer module at a distal portion of a transmission cable may convert the signal (e.g., 16 signals) into one bidirectional serial line, which may increase the frequency at which the signals are transmitted (e.g., to about 2-6 GHZ, for example 3 GHZ) through a cable to a control unit. Optionally, the electrosurgical frequency which is lower (e.g., up to about 400 MHz) than the frequency of the image sensor signals, and therefore may not interfere with the image sensor signals.

According to some embodiments, the transmission cable may be configured to transfer signals from the image sensor to a proximal control unit. According to some embodiments, the proximal control unit may be located on and/or in the vicinity of the handle of the endoscope. According to some embodiments, a decoder module may be located at the proximal end of the transmission cable. According to some embodiments, a decoder module may be located within the control unit. Optionally, a decoder module may convert the signals back to a Mobile Industry Processor Interface (MIPI) Low Voltage Differential Signaling (LVDS) protocol, Display port, HDMI (High-Definition Multimedia Interface), SDI (Serial Digital Interface), HD-SDI (High-Definition Serial Digital Interface), DVI (Digital Visual Interface), FPD-Link, or GMSL (Gigabit Multimedia Serial Link) protocol. Additionally, in accordance with some embodiments, the camera head may facilitate use of a relatively long cable connecting the camera head and the control unit. According to some embodiments, visual display unit may be located at the proximal end of the transmission cable.

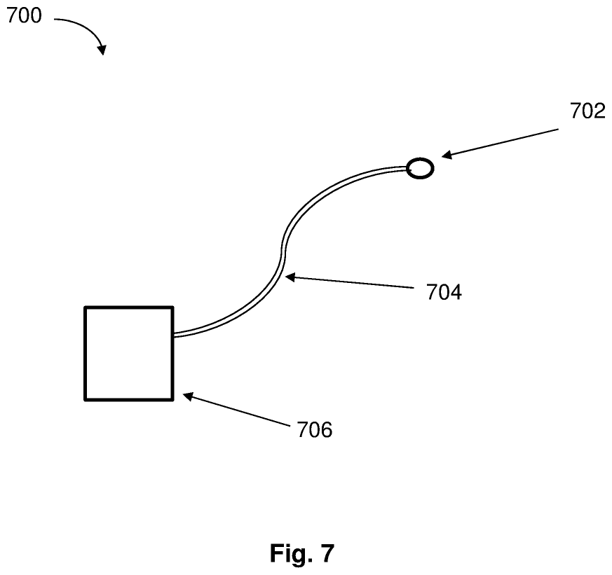

Reference is now made to FIG. 7, which shows a schematic diagram of an electrosurgical endoscopic system having a distal camera head and a proximal control and visualization unit, in accordance with some embodiments. According to some embodiments, an endoscopic system 700 includes a camera head 702 associated with a distal portion of a transmission cable 704. According to some embodiments, the transmission cable 704 is configured to transfer signals from the image sensor in camera head 702 to a proximal control unit 706. According to some embodiments, the proximal control unit 706 may be located on and/or in the vicinity of the handle of the endoscope. According to some embodiments, the transmission cable 704 may be connected to a console having a display on which the images may be displayed.

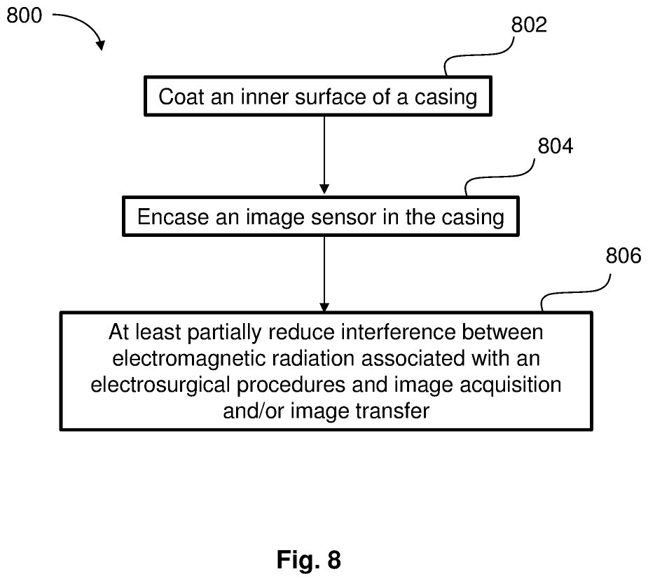

Reference is now made to FIG. 8 showing a flow diagram of a method for at least partially reducing interference between electromagnetic radiation associated with the electrosurgical procedures and image acquisition and/or image transfer in endoscopic electrosurgery procedures, in accordance with some embodiments. According to some embodiments, a method 800 for at least partially reduce interference between electromagnetic radiation associated with an electrosurgical procedures and image acquisition and/or image transfer includes coating 802 an inner surface of a camera head casing, encasing 804 at least one image sensor and at least one corresponding lens in the casing, wherein the casing is at least partially composed of a non-conductive material, thereby at least partially reduce 806 interference between electromagnetic radiation associated with an electrosurgical procedures and image acquisition and/or image transfer.

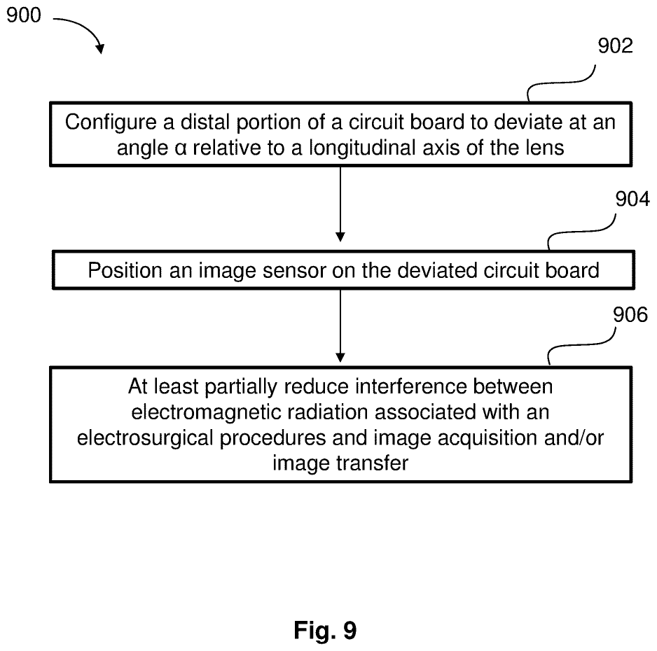

Reference is now made to FIG. 9, which shows a flow diagram of a method for at least partially reducing interference between electromagnetic radiation associated with the electrosurgical procedures and image acquisition and/or image transfer in endoscopic electrosurgery procedures, in accordance with some embodiments. According to some embodiments, a method 900 for at least partially reduce interference between electromagnetic radiation associated with an electrosurgical procedures and image acquisition and/or image transfer includes configuring 902 a distal portion of a circuit board to deviate at an angle α relative to a longitudinal axis of a lens, positioning 904 an image sensor on the deviated portion of the circuit board (e.g., PCB) associated with the image sensor at an angle α relative to a longitudinal axis defined by the center of the corresponding lens, thereby at least partially shielding 906 the image sensor from radiation generated during the electrosurgery procedure. In some embodiments, the circuit board may be composed of one or more layers. According to some embodiments, at least one of the layer of the electric circuit board may include coating, e.g., a thin metal layer, for shielding the electric circuit board from signals generated by the electrosurgical process. According to some embodiments, the angle of the electric circuit board may shield the image sensor and/or some or all the electronics further down the electric circuit board, due to the coating on the electric circuit board. According to some embodiments, the method at least partially reduce interference between electromagnetic radiation associated with an electrosurgical procedures and image acquisition and/or image transfer.

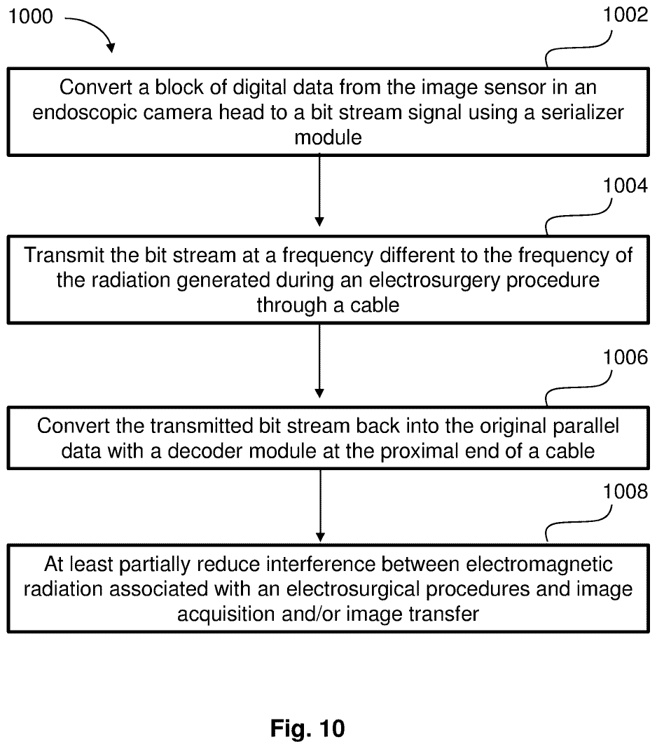

Reference is now made to FIG. 10 which shows a flow diagram of a method for at least partially reducing interference between electromagnetic radiation associated with the electrosurgical procedures and image acquisition and/or image transfer in endoscopic electrosurgery procedures, in accordance with some embodiments. According to some embodiments, the method 1000 for at least partially reduce interference between electromagnetic radiation associated with an electrosurgical procedures and image acquisition and/or image transfer includes converting 1002 a block of digital data from the image sensor in an endoscopic camera head to a bit stream signal using a serializer module. Transmitting 1004 the bit stream at a frequency different to the frequency of the radiation generated during an electrosurgery procedure through a cable, and converting 1006 the transmitted bit stream back into the original parallel data with a decoder module at the proximal end of a cable or at a control unit without any loss of information or corruption, thereby at least partially reducing 1008 interference between electromagnetic radiation associated with an electrosurgical procedures and image acquisition and/or image transfer.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 80% and 120% of the given value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

The following examples are presented to provide a more complete understanding of the invention. The specific techniques, conditions, materials, proportions and reported data set forth to illustrate the principles of the invention are exemplary and should not be construed as limiting the scope of the invention.

1.-52. (canceled)

53. A camera head for use in endoscopic electrosurgery procedures, the camera head comprising:
at least one image sensor; and
a serializer module configured to convert one or more image signals acquired by the image sensor to a serial signal configured to be transmitted at a frequency which is different to a frequency of the radiation generated during the electrosurgery procedure.

54. The camera head according to claim 53, wherein the radiation generated during the electrosurgery procedure comprises electromagnetic radiation.

55. The camera head according to claim 54, wherein the electromagnetic radiation generated during the electrosurgery procedure is a radiofrequency radiation (RF) in the range of about 0.1 MHz to about 4 MHz.

56. The camera head according to claim 53, wherein the serial signal generated by the serializer module is transmitted at a frequency in the range of about 2 GHz to about 8 GHZ.

57. The camera head according to claim 53, further associated with a distal portion of a transmission cable configured to transfer signals from the image sensor to a proximal control unit, and
a decoder module wherein the decoder module is configured to convert the transmitted serialized signal to a non-serialized signal.

58. The camera head according to claim 53, wherein the serialization of the signals by the serializer module and/or the deserialization of the signals by the module make use of a Mobile Industry Processor Interface (MIPI), a Low Voltage Differential Signaling (LVDS), Display port, HDMI (High-Definition Multimedia Interface), SDI (Serial Digital Interface), HD-SDI (High Definition Serial Digital Interface), DVI (Digital Visual Interface), FPD-Link and/or a GMSL (Gigabit Multimedia Serial Link) protocol.

59. The camera head according to claim 58, further comprising a housing at least partially composed of a non-conductive material, selected from the group consisting of: a thermoset, a thermoplastic and a ceramic material.

60. The camera head according to claim 59, further comprising a coating on a surface of the casing.

61. The camera head according to claim 60, wherein the coating is provided on an inner surface of the casing.

62. The camera head according to claim 61, wherein the coating is a layer of a conductive polymer, a metal, or a combination thereof,
wherein the metal is selected from the group consisting of copper, gold, silver, iron, aluminum, nickel, and any combination thereof.

63. The camera head according to claim 62, wherein the coating is a layer of a metal, and wherein the coating is coated onto the casing by an electroplating process.

64. The camera head according to claim 62, wherein the coating has a thickness in the range of between about 0.1 μm to about 30 μm.

65. The camera head according to claim 53, wherein the image sensor is positioned on an electronic circuit board associated with the image sensor, wherein at least a distal portion of the circuit board is configured to deviate at an angle α relative to a longitudinal axis of a corresponding lens.

66. The camera head according to claim 65, wherein angle α is in the range of about 15° to about 180°.

67. The camera head according to claim 53, further comprising at least one of an illumination source, a communication unit and a power unit or any combination thereof.

68. The camera head according to claim 53, wherein the electrosurgical procedure is selected from: cutting, coagulating, dissecting, fulgurating, ablating and/or shrinking tissue.

69. The camera head according to claim 53, further comprising, an electric circuit board associated with the image sensor, wherein the electric circuit board is coated with a conducting layer for shielding the electric circuit board from signals generated by the electrosurgical process,
wherein at least a distal portion of the electric circuit board is configured to deviate at an angle α relative to a longitudinal axis defined by the center of the corresponding lens,
wherein the angle of the image sensor is configured to at least partially prevent, interference of image acquisition and/or transfer by radiation generated during the electrosurgery procedure.

70. An endoscope comprising the camera head according to claim 53, further comprising at least one working channel for electrosurgery tools.

71. A method for at least partially preventing interference of image acquisition and/or image transferring in a camera head in an endoscopic electrosurgery procedure, the method comprising:
providing at least one image sensor configured to acquire one or more images; and
converting one or more image signals acquired by the image sensor during the endoscopic electrosurgical procedure to a serial signal using a serializer module configured for transmitting at a frequency which is different to a frequency of the radiation generated during the electrosurgery procedure,
thereby at least partially preventing interference of image acquisition and/or transfer by radiation generated during the electrosurgery procedure.

72. The method of claim 71, further comprising converting the transmitted serialized signal to a non-serialized signal using a decoder module at the proximal end of a transmission cable or at a control unit.
